(19) United States
(12) Patent Application Publication
(10) Pub. No.: US 2009/0311331 A1
(43) Pub. Date: Dec. 17, 2009

(54) Pellet Formulation Comprising Colloidal Silicon Dioxide

(76) Inventor: Gisela Fridrun Podczeck, Houghton-le-Spring (GB)

Correspondence Address: BRINKS, HOFER, GILSON & LIONE, P.O. BOX 1340, MORRISVILLE, NC, 27560, US

(21) Appl. No.: 12/306,387

(22) PCT Filed: Jun. 28, 2007
(86) PCT No.: PCT/GB07/50367

§ 371 Date: Sep. 1, 2009

(30) Foreign Application Priority Data
Jun. 28, 2006 (GB) 0612809.4

(51) Int. Cl. A61K 9/14; A61K 31/135; A61K 31/16; A61K 31/19; A61K 31/341

(52) U.S. Cl. 424/489; 514/653; 514/629; 514/570; 514/471

(57) ABSTRACT

The present invention provides a pellet formulation comprising colloidal silicon dioxide (CSD) and one or both of a surfactant and a plasticiser. A process for the production of 5 said formulation is also provided.

### FIELD OF THE INVENTION

The present invention relates to compounds and their use in pellet formulations, especially pharmaceutical formulations.

### BACKGROUND TO THE INVENTION

Several major pharmaceutical pellet formulations are prepared by an extrusion/spheronization process. The active ingredient and an excipient are mixed with a liquid (usually water) to form a paste, which is subsequently forced through a mesh to form strands. These strands are then placed onto a horizontal plate rotating in a cylinder, which causes them to break and round into spherical pellets. The pellets are dried and may be coated, e.g. with an enteric coating, if desired.

Commercial pellet formulations often include microcrystalline cellulose (MCC) as an excipient. Although some alternative excipients have been proposed, e.g. powdered cellulose (Lindner et al, J. Pharm. Pharmacol, 1994, 46, 2-7), a combination of waxes, starches and maltodextrin (Zhou et al, Int. J. Pharm., 1996, 133, 155-160), hydroxypropyl methylcellulose and hydroxyethylcellulose (Chatlapalli et al, Int. J. Pharm., 1998, 161, 179-193), β-cyclodextrin (Gazzaniga et al, Drug Dev. Ind. Pharm., 1998, 24, 869-873), pectinic acid (Tho et al, Eur. J. Pharm. Sci., 2002, 54, 95-99) chitosan (Steckel et al, Eur. J. Pharm. Biopharm., 2004, 57, 197-114), glyceryl monostearate (Newton et al, Pharm. Technol. Eur., 2004, 16 (10) 21-27) and starch-dextrin (Almeida Prieto et al, Eur. J. Pharm. Biopharm., 2005, 59, 511-521), none of these materials has established itself as an adequate replacement for MCC. While MCC is commonly used as an excipient in pellet formulations, it is incompatible with certain drugs, for example ranitidine.

Colloidal silicon dioxide (CSD) is a fumed silica prepared by vapour-phase hydrolysis of a silicon compound, such as silicon tetrachloride. The product itself is usually a submicron, fluffy, light, loose, bluish-white, odorless and tasteless amorphous powder which is commercially available from a number of sources, including Cabot Corporation (under the trade name Cab-O-Sil); Degussa, Inc. (under the trade name Aerosil); Huber Engineered Materials (Huber GL100 and GL200); Wacker (Wacker HDK®; and E.I. DuPont & Co. CSD is also known as colloidal silica, fumed silica, light anhydrous silicic acid, silicic anhydride, and silicon dioxide fumed, among others. A variety of commercial grades of CSD are produced by varying the manufacturing process.

CSD is used in tablet and capsule formulations as a glidant, i.e. a material added to improve powder flow. The amount of silicon dioxide included in tablets and capsules is normally very limited, e.g. from 0.1 to 0.5% by weight. This is partly due to the fact that increasing the amount of silicon dioxide in the powder mixture used in the manufacture of tablets and capsules may cause the mixture to flow too well, causing a varying tablet or capsule weight and an uneven content distribution.

### SUMMARY OF THE INVENTION

The present invention is based at least in part on a discovery that CSD, when combined with one or more surfactants and/or plasticisers, is particularly suitable for use as an excipient in formulations, especially pellet formulations. More particularly, it has been found that the combination of CSD with a surfactant and/or a plasticiser provides a viable alternative to MCC as a spheronising aid for pellet formulations.

Accordingly, the present invention provides a pellet formulation comprising CSD and one or both of a surfactant and a plasticiser.

Also provided is a process for the production of a pellet formulation of the invention, which comprises mixing colloidal silicon dioxide (CSD) with one or both of a surfactant and a plasticiser, and forming one or more pellets from the resulting mixture.

In particular, the formulation may be obtained by an extrusion/spheronization process. This may involve forming a wet mass or paste by mixing the CSD and an aqueous solution containing a surfactant and/or a plasticiser, optionally with one or more other ingredients, for example selected from a therapeutic agent, a diagnostic agent, a herbicide, a pesticide, a fertiliser, animal feed. The wet mass may also comprise one or more other ingredients, for example selected from a filler, a disintegrant, a preservative, a stabiliser, an antioxidant and a binder. The wet mass can then be extruded using a mesh or screen, or a long die system (e.g. a ram, rotating cylinder or rotating gear). The extruded material can then be spheronized and dried if necessary, to produce one or more pellets, which can be coated and/or loaded into a capsule for example.

Formulations of the invention may be pharmaceutical formulations, or may be suitable for use in agriculture or horticulture. For example, the formulations may be useful in the delivery of therapeutic agents (including vaccines), diagnostic agents, herbicides, pesticides, fertilizers and animal feed. Pharmaceutical formulations of the invention may be capable of releasing their drug content more rapidly compared with equivalent MCC-based formulations; pellets containing CSD have been found to have a faster rate of disintegration in water compared with MCC.

### DESCRIPTION OF VARIOUS EMBODIMENTS

The present invention provides a pellet formulation containing CSD in combination with a surfactant and/or a plasticiser. The formulation may be a pharmaceutical formulation. The pellets may be used to produce a solid formulation, such as a capsule or package.

CSD may be present in various forms, including hydrophilic and hydrophobic forms. The CSD may comprise particles having a size of from about 1 nanometer (nm) to about 100 microns (μm), based on average primary particle size, the excipient having a moisture content of from about 0.5 to 2.5% loss-on drying (LOD), particularly between about 0.5 and about 1.8% LOD, more particularly between 0.8 and 1.5% LOD, and especially between about 0.8 and about 1.2% LOD.

The surface area of the CSD may range from about 50 m²/gm to about 500 m²/gm. The average primary particle diameter may ranges from about 5 nm to about 50 nm. In commercial colloidal silicon dioxide products, these particles are agglomerated or aggregated to varying extents. The bulk density of the CSD may range from about 20 g/l to about 100 g/l.

The CSD may be in light or dense form. Light CSD typically has a tapped density value of from about 40 to about 80 g/l (e.g. from about 50 to about 60 g/l), whereas dense CSD typically has a tapped density of from about 80 to about 200 g/l (e.g. from about 90 to about 120 g/l). Examples of light CSD include Cab-O-Sil M-5 (36.8 g/l, Cabot) and Cab-O-Sil S-17 (72 g/l, Cabot). Examples of dense CSD include Aerosil 200VV (134 g/l, Degussa), Aerosil 130VV (118 g/l, Degussa, Aerosil R972V (115 g/l, Degussa), Aerosil R974V (105 g/l, Degussa), Cab-O-Sil M-7D (100 g/l, Cabot), Wacker HDK H2000 (220 g/l), H2015 (200 g/l) and H2050 (200 g/l).

Commercially available CSD products have, for example, a BET surface area ranging from about 50±15 m²/gm (Aerosil OX50) to about 400±20 m²/gm (Cab-O-Sil S-17) or 390±−40 m²/gm (Cab-O-Sil EH-5). Commercially available particle sizes range from a nominal particle diameter of 7 nm (e.g., Cab-O-Sil S-17 or Cab-O-Sil EH-5) to an average primary particle size of 40 nm (Aerosil OX50). The pH of the these products at 4% aqueous dispersion ranges from pH 3.5 to pH 4.5. These commercially available products are described for the purpose of exemplifying properties of commercially available CSD materials, and should not be construed as limiting.

Of mention are formulations substantially free of MCC, especially formulations in which MCC is absent.

The formulation may comprise one or more surfactants (i.e. surface-active agents), which may be selected from non-ionic, ionic and ampholytic surfactants. Examples of non-ionic surfactants include cremophores (e.g. Cremophor ELP®, pluronic compounds, Tween compounds (e.g. Tween 80®, and mixtures thereof. An ionic surfactant may be an anionic or cationic surfactant. Examples of anionic surfactants include sodium laurylsulphate, fatty acid soaps, alkylsulfonates, alkylphosphates, ether phosphates, fatty acid salts of basic amino acids; triethanolamine soap, and alkyl quaternary ammonium salts while an exemplary cationic surfactant is cetrimide. Ampholytic surfactants include lecithins, betaines and aminocarboxylic acid salts. The formulation may comprise an aqueous solution of the surfactant.

The formulation may comprise one or more plasticisers, for example glycerides, e.g. monoglycerides, diglycerides, triglycerides, or mixtures thereof.

The formulation may comprise one or more of sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; and bile acid and salts thereof (e.g. chenodeoxycholic acid, cholic acid, deoxycholic acid, dehydrocholic acid and salts thereof, and glycine or taurine conjugate thereof).

The formulation may comprise a surfactant and a plasticiser. Of particular mention are formulation obtained using aqueous solutions containing the surfactant and one or more of the above plasticisers, especially glycerides.

The formulation is in pellet (or spheronised) form, i.e. the formulation comprises one or more pellets comprising CSD and a surfactant and/or plasticiser. The formulation may comprise a plurality of pellets. In one embodiment, the or each pellet is substantially spheroidal. By way of example, the or each pellet may have a diameter of from about 0.1 mm to about 5.0 mm, e.g. from about 0.5 mm to about 5.0 mm, in particular from about 0.5 mm to about 2.5 mm, more particularly from about 0.8 to about 1.5 mm. The pellets may be used in the production of extrudates, capsules, tablets, powders, granules and the like.

A pellet formulation of the invention may be obtained by mixing CSD with a surfactant and/or a plasticiser, and forming one or pellets from the resulting mixture. The properties of the pellet formulation can be optimised by varying the quantities of CSD and surfactant and/or plasticiser. Suitable proportions of components are illustrated, without limitation, in the Examples given herein. In embodiments, CSD is used in an amount of at least 1% by weight, for example from about 5 to about 50% by weight, in particular from about 10 to about 40% by weight.

The formulation may be produced using any suitable technique known in the art, such techniques including extrusion/spheronization, rotating pan, centrifugal rotary processing, fluid bed agglomeration, granulation/spheronisation and direct spheronisation.

In particular, the formulation may be obtained by an extrusion/spheronization process. Extrusion/spheronization generally involves the steps of dry blending the dry ingredients (e.g. drug and excipients), wet granulation of the dry blend and extrusion of the wet mass through a mesh to produce compacted cylindrical strands, and spheronization of the strands in a spheronizer. Typically, a dry blend of the composition is first prepared. Water is then added slowly, with continuous mixing until a granulation of the requisite consistency is obtained. Alternatively, the drug or other active ingredient, if it is water soluble, can be dissolved is in the water, and this solution added to the particulate composition. The wet granulation is extruded through suitably sized mesh and spheronized using a rotating disk having a ground surface. A spheronization device typically consists of a hollow cylinder with a horizontal rotating plate, which is normally grooved or serrated. The filaments are broken in short segments which are transformed in spherical or quasi-spherical particles on the upper surface of the rotating plate at a velocity typically ranging from about 200 rpm to about 2000 rpm. It will be appreciated that the velocity required may be lesser or greater than these exemplary values, and will generally depend on the spheronizer plate, size and design, as well as loading. Under the tumbling/roping like action of the rotating disk, the cylindrical strands are broken into smaller segments which undergo smoothing and rounding to form the spheroids which are then dried. The spheronized particles may be dried in any suitable way, such as for example the air drying or in a static condition or their combination. In particular, the spheres may be dried in a fluidized bed or conventional oven, usually to a moisture level of about 0.5% to about 5%. The particles are used as they are or they are coated to obtain granules for use in tablets, capsules, packets and other formulations.

The formulation may comprise one or more coatings or shells, such as enteric coatings and other coatings well known in the art. They may optionally contain opacifying agents and may also be of a composition such that they release the active ingredient only, or preferentially, in a certain part of the intestinal tract, and/or in delayed fashion. The formulation may be a non-pareil formulation in which the active ingredient is coated on a core comprising the CSD and surfactant.

The formulation may comprise a hydrophilic coating or a hydrophobic coating. An example of a material suitable for use as a hydrophilic coating is hydroxypropylmethylcellulose (e.g., Opadry®, commercially available from Colorcon, West Point, Pa.; and Kollicoat SR30). Materials useful as hydrophobic coatings include derivatives of acrylic acid (such as esters of acrylic acid, methacrylic acid, and copolymers thereof) celluloses and derivatives thereof (such as ethylcellulose), polyvinylalcohols, and the like.

The coatings may be applied in any manner known to those skilled in the art. For example, in one embodiment, the coating is applied via a fluidized bed or in a coating pan.

The formulation may further comprise one or more of: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents including inorganic compound such as iron oxides, barium sulphate and calcium carbonate; e) solution retarding agents such as waxes; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. The formulation may further comprise a buffering agent.

The formulation is especially a pharmaceutical formulation. A formulation of the invention may therefore comprise one or more pharmaceutically active ingredients. A pharmaceutical formulation will typically in solid form. Formulations for oral administration are of particular mention. The pellets may be used to produce pharmaceutical formulations, examples including capsules, tablets, powders, and granules. The formulation may be a pareil or non-pareil formulation.

For a pharmaceutical formulation, actual dosage levels of the active ingredient(s) in the formulation may be varied so as to obtain an amount of the active ingredient(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

An exemplary dosage level of active ingredient is from about 0.01 to about 500 mg per kg patient body weight per day which can be administered in single or multiple doses. In particular, the dosage level may be from about 0.1 to about 250 mg/kg per day, e.g. about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the formulations may contain 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of the active ingredient for the symptomatic adjustment of the dosage to the patient to be treated. The formulation may be administered on a regime of 1 to 4 times per day, preferably once or twice per day. The dosage regime may be adjusted to provide the optimal therapeutic response.

The formulation may comprise one or more active ingredients selected from systemically active therapeutic agents, locally active therapeutic agents, disinfecting agents, chemical impregnants, cleansing agents, deodorants, fragrances, dyes, animal repellents, insect repellents, fertilizing agents, pesticides, herbicides, fungicides, and plant growth stimulants, and the like. The proportion of active ingredient present in the formulation may range from about 0.001% to about 90%, especially from about 0.1% to about 60%.

A wide variety of therapeutic agents can be used in conjunction with the present invention. The therapeutic agents (e.g. pharmaceutical agents) used in the compositions of the present invention may include both water soluble and water insoluble drugs. Examples of suitable agents include antihistamines (e.g. dimenhydrinate, diphenhydramine, chlorpheniramine and dexchlorpheniramine maleate), analgesics (e.g. aspirin, codeine, morphine, dihydromorphine, oxycodone, etc.), non-steroidal anti-inflammatory agents (e.g. naproxen, diclofenac, indomethacin, ibuprofen, sulindac), anti-emetics (e.g. metoclopramide), anti-epileptics (e.g. phenytoin, meprobamate and nitrazepam), vasodilators (e.g. nifedipine, papaverine, diltiazem and nicardirine), anti-tussive agents and expectorants (e.g. codeine phosphate), anti-asthmatics. (e.g. theophylline), antacids, anti-spasmodics (e.g. atropine, scopolamine), antidiabetics (e.g. insulin), diuretics (e.g. ethacrynic acid, bendrofluazide), anti-hypotensives (e.g. propranolol, clonidine), antihypertensives (e.g clonidine, methyldopa), bronchodilators (e.g. albuterol), steroids (e.g. hydrocortisone, triamcinolone, prednisone), antibiotics (e.g. tetracycline), antihemorrhoidals, hypnotics, psychotropics, antidiarrheals, mucolytics, sedatives, decongestants, laxatives, vitamins, stimulants (including appetite suppressants such as phenylpropanolamine). Of particular mention as an active ingredient is ephedrine hydrochloride, which may be used as a nasal decongestant, or in the treatment of coughs and colds. Also of mention are paracetamol, ibuprofen and ranitidine.

A wide variety of locally active agents can be used, including both water soluble and water insoluble agents. The locally active agent(s) which may be included in the controlled release formulation of the present invention is intended to exert its effect in the environment of use, e.g. the oral cavity, although in some instances the active agent may also have systemic activity via absorption into the blood via the surrounding mucosa. Locally active agents include antifungal agents (e.g. amphotericin B, clotrimazole, nystatin, ketoconazole, miconazol, etc.), antibiotic agents (penicillins, cephalosporins, erythromycin, tetracycline, aminoglycosides, etc.), antiviral agents (e.g acyclovir, idoxuridine, etc.), breath fresheners (e.g. chlorophyll), antitussive agents (e.g. dextromethorphan hydrochloride), anti-cariogenic compounds (e.g. metallic salts of fluoride, sodium monofluorophosphate, stannous fluoride, amine fluorides), analgesic agents (e.g. methylsalicylate, salicylic acid, etc.), local anesthetics (e.g. benzocaine), oral antiseptics (e.g. chlorhexidine and salts thereof, hexylresorcinol, dequalinium chloride, cetylpyridinium chloride), anti-flammatory agents (e.g. dexamethasone, beta-methasone, prednisone, prednisone, triamcinolone, hydrocortisone, etc.), hormonal agents (oestriol), antiplaque agents (e.g chlorhexidine and salts thereof, octenidine, and mixtures of thymol, menthol, methysalicylate, eucalyptol), acidity reducing agents (e.g. buffering agents such as potassium phosphate dibasic, calcium carbonate, sodium bicarbonate, sodium and potassium hydroxide, etc.), and tooth desensitizers (e.g. potassium nitrate). Formulations of the invention may also include other locally active agents, such as flavorants and sweeteners.

The following Examples illustrate the invention.

### EXAMPLE 1

The following ingredients were mixed to form a paste:

| Component | Amount |
|---|---|
| Colloidal silicon dioxide | 25 g |
| 5% solution of equal parts of | 60 g |
| Tween 80 and a mixture of mono | |
| and diglycerides in water | |

The mixture was then extruded through a 1 mm diameter die, 4 mm in length, spheronised at 500 rpm for 5 minutes on a 12.5 cm plate and dried to constant weight at 60° C. to produce a pellet formulation.

### EXAMPLE 2

The following ingredients were mixed to form a paste:

| Component | Amount |
|---|---|
| Colloidal silicon dioxide | 20 g |
| Magnesium carbonate | 20 g |
| Mixture of mono and diglycerides | 3 g |
| Ephedrine hydrochloride | 20 g |
| Water | 45.6 g |

The mixture was then extruded through a 1.5 mm screen, spheronised on a 12.5 cm plate at low speed, and dried to constant weight to produce a pellet formulation.

### EXAMPLE 3

A pellet formulation containing the following components was prepared according to the procedure described in Example 2:

| Component | Amount |
|---|---|
| Colloidal silicon dioxide | 20 g |
| Lactose monohydrate | 20 g |
| Cremophor ELP | 2.08 g |
| Mixture of mono and diglycerides | 3.0 g |
| Ephedrine hydrochloride | 20 g |
| Water | 41.6 g |

### EXAMPLE 4

A pellet formulation containing the following components was prepared according to the procedure described in Example 2.

| Component | Amount |
|---|---|
| Colloidal silicon dioxide | 20 g |
| Magnesium carbonate | 20 g |
| Cremophor ELP | 2.28 g |
| Ephedrine hydrochloride | 20 g |
| Water | 45.6 g |

### EXAMPLE 5

Various CSD-containing pellet formulations were produced. The starting materials and methods used to make each formulation are summarised in the table below:

| | CSD | Drug | Mg-Carb | Lactose | Imwitor | | 5% CMP solution | |
|---|---|---|---|---|---|---|---|---|
| No. | (g) | (g) | (g) | (g) | 742 (g) | Water (g) | (g) | Extruder (speed) |
| 1 | 20 | 20 | 20 | — | — | — | 45.6 | radial screen (4) |
| 2 | 20 | 20 | 20 | — | 3 | 46.6 | — | radial screen (4) |
| 3 | 20 | 20 | 20 | — | 3 | — | 45.6 | radial screen (4) |
| 4 | 20 | 20 | — | 20 | — | — | 40.6 | radial screen (3) |
| 5 | 20 | 20 | — | 20 | 3 | — | 41.6 | radial screen (3) |
| 6 | 20 | — | 40 | — | — | — | 59.6 | radial screen (5) |
| 7 | 20 | — | 40 | — | 3 | 58.6 | — | radial screen (5) |
| 8 | 20 | — | 40 | — | 3 | — | 58.6 | radial screen (5) |
| 9 | 20 | — | — | 40 | — | — | 45.6 | radial screen (4) |
| 10 | 20 | — | — | 40 | 3 | — | 46.6 | radial screen (4) |
| 11 | 20 | 40 | — | — | — | — | 37.6 | radial screen (3) |
| 12 | 20 | 40 | — | — | 3 | — | 37.6 | radial screen (3) |
| 13 | 60 | — | — | — | — | — | 127.1 | radial screen (5) |
| 14 | 60 | — | — | — | 3 | — | 123 | radial screen (5) |
| 15 | 36.5 | 36.5 | — | — | 2.3 | — | 87.7 | ram |
| 16 | 10 | 30 | — | — | 0.6 | — | 23.4 | ram |
| 17 | 10 | 30 | — | — | 0.6 | — | 23.4 | ram |
| 18 | 25 | — | — | — | 1.5 | — | 60 | ram |

Hydrophilic grade CSD (Aerosil 200®, Degussa) was used in each formulation. The lactose was lactose monohydrate of BP grade. The surfactant comprised a CMP solution comprising 5% of Cremophor ELP (BASF) in distilled water. Imwitor 742® was used as a plasticiser. The drug used in Formulations 1 to 5, 11 and 12 was ephedrine hydrochloride. In Formulations 15 and 16, the drug was paracetamol, while in Formulation 17 ibuprofen was used.

The spheronizer used in each case comprised a 12.5 cm cross-hatch plate at low speed settings. The spheroniser speed shown in the above table is as measured in equipment units. The spheronizing time for Formulations 1 to 14 was 10 minutes. For Formulations 15 to 18, the spheronizing time was 5 minutes. A 1.5 mm radial screen and a ram comprising a long die of 1 mm diameter and 4 mm length were used.

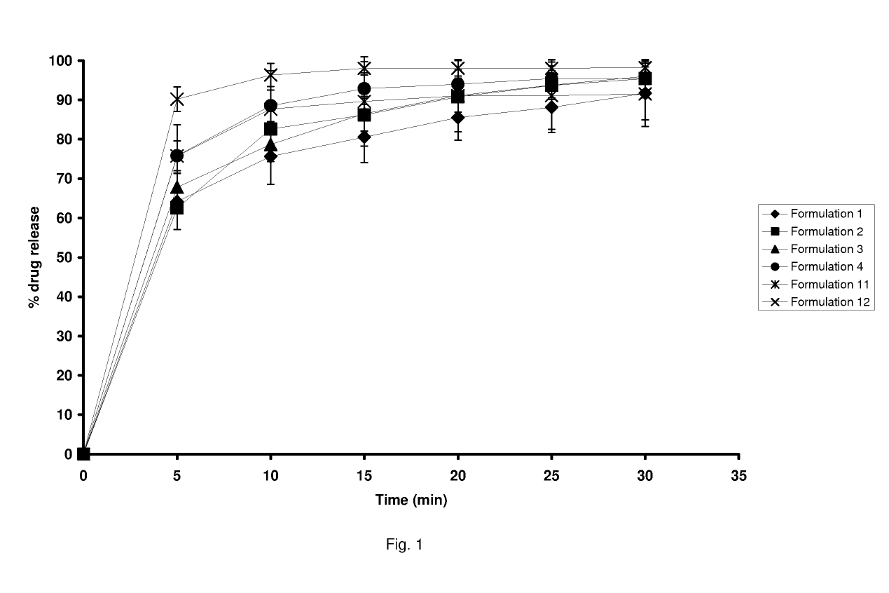

The properties of each pellet formulation are summarised in the table below. The dissolution profile of Formulations 1, 2, 3, 4, 11 and 12 is shown in FIG. 1. The time taken to achieve 70% dissolution was no more than 10 minutes for each formulation. The common standard of BP 2005 and all other European, US and Japanese pharmacopoeia is a maximum of 45 minutes for 70% dissolution.

| | Sieve Analysis - % yield in | | | Tensile | | | Image Analysis | |
|---|---|---|---|---|---|---|---|---|
| | fraction | | | Strength | Density | Porosity | | Feret Diameter |
| No. | 0.71-1.0 mm | 1.0-1.4 mm | 1.4-2.0 mm | (Mpa) | (g/cm³) | (%) | Aspect ratio | (μm) |
| 1 | 4.6 | 47.8 | 46.8 | 0.34 ± 0.08 | 1.46 ± 0.01 | 20.3 | 1.17 ± 0.14 | 1226 ± 113 |
| 2 | 5.8 | 76.4 | 17.4 | 0.24 ± 0.05 | 1.51 ± 0.03 | 17.2 | 1.16 ± 0.11 | 1280 ± 115 |
| 3 | 4.3 | 45.5 | 48.8 | 0.24 ± 0.06 | 1.58 ± 0.01 | 12.1 | 1.16 ± 0.14 | 1290 ± 110 |
| 4 | <1 | 5.2 | 55.6 | 0.40 ± 0.13 | 1.46 ± 0.01 | 13.8 | 1.21 ± 0.12 | 1428 ± 147 |
| 5 | <1 | 16 | 72.9 | 0.27 ± 0.07 | 1.45 ± 0.01 | 12.2 | 1.27 ± 0.15 | 1288 ± 191 |
| 6 | 4.6 | 61.7 | 32.6 | <0.1 | 1.86 ± 0.02 | 8.1 | 1.13 ± 0.12 | 1310 ± 120 |
| 7 | 17.6 | 63.8 | 16.2 | <0.1 | 1.89 ± 0.04 | 6.3 | 1.11 ± 0.14 | 1321 ± 122 |
| 8 | 14.7 | 65.9 | 17.7 | <0.1 | 1.86 ± 0.03 | 4.8 | 1.11 ± 0.13 | 1305 ± 130 |
| 9 | 12.7 | 61.4 | 25.3 | 0.25 ± 0.07 | 1.65 ± 0.02 | 4.7 | 1.13 ± 0.13 | 1324 ± 122 |
| 10 | 34.3 | 52.8 | 7.4 | <0.1 | 1.56 ± 0.01 | 7.8 | 1.11 ± 0.14 | 1236 ± 116 |
| 11 | 8.4 | 34.9 | 51.5 | 0.37 ± 0.09 | 1.30 ± 0.01 | 21.2 | 1.18 ± 0.14 | 1414 ± 140 |
| 12 | 7.7 | 34.2 | 51.6 | 0.22 ± 0.04 | 1.36 ± 0.00 | 16 | 1.15 ± 0.13 | 1312 ± 143 |
| 13 | 59.2 | 16.7 | <5 | <0.1 | 2.00 ± 0.06 | 4.4 | 1.22 ± 0.19 | 1227 ± 200 |
| 14 | 54.3 | 6.6 | <5 | <0.1 | 1.74 ± 0.04 | 14.9 | 1.21 ± 0.20 | 1238 ± 203 |
| 15 | <1 | >90 | <5 | 0.21 ± 0.02 | 1.50 ± 0.02 | 13.5 | 1.05 ± 0.12 | 1221 ± 106 |
| 16 | <1 | >90 | <5 | 0.25 ± 0.03 | 1.46 ± 0.03 | 7.6 | 1.07 ± 0.13 | 1195 ± 97 |
| 17 | <1 | >90 | <5 | 0.22 ± 0.05 | 1.48 ± 0.03 | 5.7 | 1.06 ± 0.11 | 1179 ± 86 |
| 18 | <1 | >90 | <5 | 0.12 ± 0.07 | 1.92 ± 0.07 | 4.6 | 1.11 ± 0.18 | 1205 ± 103 |

1-43. (canceled)

44. A pellet formulation comprising, colloidal silicon dioxide (CSD) and one or both of a surfactant and a plasticiser.

45. The formulation of claim 44, wherein said formulation comprises one or more pellets wherein said one or more pellets has a diameter of from about 0.5 mm to about 5.0 mm.

46. The formulation of claim 45, wherein said one or more pellets has a diameter of from about 0.5 mm to about 2.5 mm.

47. The formulation of claim 44, which comprises a surfactant.

48. The formulation of claim 47, which comprises a non-ionic surfactant.

49. The formulation of claim 48, which comprises a cremophore or a pluronic compound.

50. The formulation of claim 49, which comprises CREMOPHOR ELP®.

51. The formulation of claim 48, which comprises TWEEN 80®.

52. The formulation of claim 47, which comprises an ionic surfactant.

53. The formulation of claim 52, which comprises an anionic surfactant or a cationic surfactant.

54. The formulation of claim 53, wherein said anionic surfactant is sodium laurylsulphate.

55. The formulation of claim 53, wherein the cationic surfactant is cetrimide.

56. The formulation of claim 47, which comprises an ampholytic surfactant.

57. The formulation of claim 56, which comprises a lecithin.

58. The formulation of claim 44, which comprises a plasticiser.

59. The formulation of claim 58, which comprises a glyceride.

60. The formulation of claim 59, wherein said glyceride is a monoglyceride or diglyceride or a mixture thereof.

61. The formulation of claim 44, which comprises a surfactant and a plasticiser.

62. The formulation of claim 44, wherein said CSD is hydrophilic or hydrophobic.

63. The formulation of claim 44, which further comprises an excipient.

64. The formulation of claim 63, wherein said excipient is magnesium carbonate or lactose monohydrate.

65. The formulation of claim 44, which is substantially free of microcrystalline cellulose (MCC).

66. The formulation of claim 44, which further comprises a therapeutic agent.

67. The formulation of claim 66, wherein said therapeutic agent is selected from the group consisting of ephedrine hydrochloride, paracetamol, ibuprofen and ranitidine.

68. The formulation of claim 44, which further comprises a diagnostic agent, a herbicide, a pesticide, a fertiliser or animal feed.

69. A process for producing the pellet formulation of claim 44, which comprises mixing colloidal silicon dioxide (CSD) with one or both of the group consisting of a surfactant and a plasticiser, and forming one or more pellets from the resulting mixture.

70. The process of claim 69, which comprises mixing said CSD and said surfactant and/or plasticiser with one or more ingredients selected from the group consisting of a therapeutic agent, a diagnostic agent, a herbicide, a pesticide, a fertiliser and animal feed.

71. The process of claim 69, which comprises mixing said CSD and said surfactant and/or plasticiser with one or more ingredients selected from the group consisting of a filler, a disintegrant, a preservative, a stabiliser, an antioxidant and a binder.

72. The process of claim 69, which comprises mixing said CSD and a solution containing said surfactant and/or plasticiser, to form a wet mass or paste.

73. The process of claim 72, wherein said solution containing said surfactant and/or plasticizer is an aqueous solution.

74. The process of claim 72, which further comprises extruding the wet mass or paste.

75. A process of claim 74, wherein said wet mass or paste is extruded using a mesh or screen, or a long die system.

76. The process of claim 75, wherein said long die system is selected from the group consisting of a ram, rotating cylinder and a rotating gear.

77. The process of claim 74, which further comprises spheronising said extruded material to form one or more pellets.

78. The process according to claim 69, which further comprises applying a coating to the pellets.
